(19) United States
(12) Patent Application Publication
(10) Pub. No.: US 2018/0127633 A1
(43) Pub. Date: May 10, 2018

(54) MICROENCAPSULATED ENZYMES

(71) Applicant: BASF SE, Ludwigshafen (DE)

(72) Inventors: Ewelina BURAKOWSKA-MEISE, Reichenbach (DE); Patrick LEIBACH, Landau (DE); Yun HAN, San Diego, CA (US); Dongmei REN, San Diego, CA (US)

(21) Appl. No.: 15/570,135

(22) PCT Filed: Apr. 25, 2016
(86) PCT No.: PCT/EP2016/059144

§ 371 Date: Oct. 27, 2017

Related U.S. Application Data
(60) Provisional application No. 62/154,756, filed on Apr. 30, 2015.

(51) Int. Cl. C09K 8/035; E21B 43/267; C09K 8/68; C09K 8/70; C09K 8/80; C08L 71/08; C12N 9/42

(52) U.S. Cl. CPC C09K 8/035; E21B 43/267; C09K 8/685; C09K 8/706; C12Y 302/01004; C08L 71/08; C12N 9/2437; C09K 2208/24; C09K 8/80

(57) ABSTRACT

The present disclosure relates to microcapsules with shell and a core with an average particle size of the microcapsules in the range from 0.5 to 20 μm, wherein the shell is a polyester and wherein the core material comprises an enzyme, a method of making the microcapsules and methods of using the microcapsules in the field of recovery of hydrocarbons from a subterranean formation.

The present disclosure relates to microcapsules with a shell and a core with an average particle size of the microcapsules in the range from 0.5 to 20 μm, wherein the shell is a polyester and wherein the core material comprises an enzyme.

It further relates to a method of making the microcapsules and methods of using the microcapsules in the field of recovery of hydrocarbons from a subterranean formation, for example, to reduce the viscosity of gelled fluids in a controlled manner.

It is the aim of hydraulic fracturing to increase the production of oil and/or gas from subterranean formations. Hydraulic fracturing is accomplished by injecting a pressurized fluid, commonly referred to as fracturing fluids, into a subterranean formation at pressures capable of forming fractures in the surrounding earth. Gel or hybrid fracturing fluids can contain a solvent, a gelling agent (viscosifier), proppant, and a breaker. The viscosity of the gelling agent (viscosifier) allows suspension of the proppant within the fluid and a reduced tendency of the proppant settling out during delivery into the rock formation. Examples of viscosifiers comprise biopolymers or modified biopolymers such as xanthans, Scleroglucane, galactomannan gums, cellulose derivatives such as hydroxyethylcellulose, carboxyethylcellulose or carboxymethylcellulose. It is the aim of the breaker to reduce the viscosity of the fracturing fluid after the process of fracturing in order to facilitate removal of the fracturing fluid from the subterranean formation because viscous fracturing fluid remaining in the formation may plug the formation thus reducing the production of oil. Examples of breakers comprise oxidizing agents and enzymes capable of cleaving bonds in the polymer chain.

Fracturing subterranean formations requires coordination between the gelling agent (viscosifier) and the breaker. Known techniques can be unreliable and result in premature breaking of the gelled fracturing fluid before the fracturing process is complete, and/or incomplete breaking of the gelled fracturing fluid. Premature breaking can cause a decrease in the number of fractures, desired size and geometry of the fractures obtained and proper proppant placement, thus decreasing the potential amount of hydrocarbon recovery. In addition, incomplete breaking can cause a decrease in the well conductivity and thus, the amount of hydrocarbon recovery.

Enzymes have been used as effective and environmental friendly breakers in recovery of hydrocarbons (e.g., recovery oil, natural gas, etc.) from a subterranean formation. However, the applications of enzyme breakers in hydrocarbon recovery have been limited by, for example, loss of enzymatic activity in the alkaline pH environment or high temperature environment of the fracturing liquid and/or at downhole conditions. There is a need for chemically and physically protected enzymes to allow effective break of gelled liquids (e.g., fracturing fluid) at downhole conditions.

US 2006/0205608 teaches a method of degrading a filter cake comprising an acid-soluble portion and a polymeric portion in a subterranean formation comprising the steps of: introducing a filter cake degradation composition comprising a delayed-release acid component and a delayed-release oxidizer component to a well bore penetrating the subterranean formation; allowing the delayed-release acid component to release an acid derivative and the delayed-release oxidizer component to release an acid-consuming component. The delayed-release acid component may comprise an esterase enzyme if desired. Encapsulating the delayed-release oxidizer component may be accomplished by a fluidized bed-coating process. The coating material used to encapsulate the delayed-release oxidizer component may be a resin material like a hydrolyzed acrylic resin or a degradable polymer like polyester.

WO 2015/039032 teaches particles for well treatment comprising a core and a shell, wherein the core comprises an acidifying agent and an enzyme. According to this teaching an enzyme affixed to ammonium sulfate carrier is coated with an acrylic polymer. As further coating material polyester are mentioned among others. Such particles are macroscopic and have a size from 0.25 to 2.8 mm. Macroscopic particles are neither very tight nor physically stable. They may burst easily under the shearing conditions of fracturing.

WO 97/24179 A1 discloses particles having a hydrophilic core within a shell which preferably comprises polyamides. The particles are made using interfacial condensation polymerization.

It was an object of the present invention to develop an enzyme formulation which shows a better physical stability and which sets the enzyme free after a certain time period in the subterranean formations. In particular, it was an object of the present invention that the enzyme is set free within a time frame of 30 minutes to 180 minutes. Moreover, it was an object of the present invention, that the formed microcapsules had a size in the μm range from 1 to 50—ideally below 20 μm. It is believed that the smaller capsules can better penetrate the fractures and hence their uniform distribution within the fractures can be achieved.

Accordingly, the microcapsules with a shell and a core with an average particle size of the microcapsules in the range from 0.5 to 20 μm, wherein the shell is a polyester and wherein the core material comprises an enzyme have been developed, as well as a way of producing these microcapsules and their use. Also disclosed herein are methods for making and using the microcapsules for treating subterranean formulation.

The use of polyester shell is particularly advantageous. The thermal hydrolysis of the polyester at high temperature subterranean environment provides a release mechanism for the enzyme. The complete hydrolysis of the polymer leaves no solid residual, which can further benefit the hydrocarbon recovery. The hydrolysis of polyester into acid also provides environmental acidification which allows the enzyme to be more effective.

Microcapsules

The microcapsules according to the invention comprise a capsule core and a capsule shell. The capsule core consists predominantly, to more than 95% by weight, of the core material, which may be an individual substance or a substance mixture.

As a rule the shell substantially covers the entire surface area of the enzyme-containing core. Depending on the thickness of the capsule shell, which might be influenced by the chosen process conditions and also amounts of the feed materials, the permeability of the capsules shell can be influenced to be impermeable or sparingly permeable for the capsule core material.

The average particle size (number average measured by optical microscopy) of the microcapsules is in the range from 0.5 to 20 μm. In particular the average particle size of the microcapsules is 0.5 to 15 μm, in particular 1 to 10 μm.

The microcapsules are spherical. So, the particle size cited above refers to the diameter of the microcapsules. For the skilled artisan it is self evident that in practice the particles may not necessarily have an ideal spherical shape but there may be slight differences from an ideal spherical shape. However, such differences are considered by referring to the average particle size as mentioned above. The number average should be determined by measuring the diameter of a statistically significant number of microcapsules, e.g of about 100 microcapsules.

The weight ratio of microcapsule core to microcapsule shell is generally from 50:50 to 98:2. Preference is given to a core/shell ratio of 75:25 to 97:3.

Shell

According to the invention the microcapsules comprise a shell wherein the shell is a polyester. Polyester is a category of polymers which contain ester functional groups in their main chain. Polyester is a polymer whose monomer units are linked together by ester bonds. Synthesis of polyesters is generally achieved by a polycondensation reaction of monomers bearing carboxyl and monomers bearing hydroxyl groups or monomers bearing —COX functional groups and hydroxyl functional groups, (an organic acid or organic acid halides monomers and an alcohol monomers are used therefore).

Preference is given to polyesters which are built by AA/BB-Polycondensation of two complementary monomers, for example a diol and a dicarboxylic acid/dicarboxylic acid halide. In particular the polyester is built by polycondensation of at least one alcohol selected from the group consisting of diols and polyols and at least one acid-component selected from the group consisting of divalent carboxylic acids, multivalent carboxylic acids, acid halides of a divalent carboxylic acid and acid halides of multivalent carboxylic acid.

Alcohols with two or more hydroxyl groups are referred to as diols and polyols. Polyol is to be understood as alcohol with three, four, five or more hydroxyl groups.

Preferred are di- or polyols which have 2 to 20 carbon atoms, preferably 2 to 12 carbon atoms and at least two hydroxyl groups, preferably two to five hydroxyl groups, such as ethylene glycol, diethylene glycol, triethylene glycol, 1,2-propylene glycol, 1,3-propylene glycol, 1,1-dimethyl-1,2-ethanediol, dipropylene glycol, triethylene glycol, tetraethylene glycol, pentaethylene glycol, tripropylene glycol, 1,2-, 1,3- or 1,4-butanediol, 1,5-pentanediol, neopentyl glycol, 1,6-hexanediol, 2-methyl-1,5-pentanediol, 2-ethyl-1,4-butanediol, 1,4-dimethylolcyclohexane, 2,2-bis(4-hydroxycyclohexyl)propane, glycerol, trimethylolethane, trimethylolpropane, trimethylolbutane, 2,2-bis(hydroxylmethyl)-1,3-propanediol (pentaerythritol), ditrimethylolpropane, erythritol and sorbitol.

According to a further embodiment preferred polyols are oligomeric or polymeric polyols with a degree of polymerization (DP) in the range from 10 to 6000. Preferred polymeric polyols are polyvinylalcohols.

The degree of polymerization (DP) is defined as the number average of monomeric units in polymer or oligomer. DP equals to (Mₙ/M₀) where Mₙ is the number-average molecular weight (determined by Gel-Permeation-Chromatography) and M₀ is the molecular weight of the monomer unit.

Polyvinyl alcohol (=PVA) corresponds in general according to formula

—CH₂—CHOH—CH₂—CHOH—

with low amounts (up to 2%) of the formula

—CH₂—CHOH—CHOH—CH₂—

It is known in the art that polyvinyl alcohol is produced by hydrolysis (deacetylation) of polyvinyl acetate, whereby the ester groups of polyvinyl acetate are hydrolysed into hydroxyl groups, thus forming polyvinyl alcohol. The hydrolysis may be complete or incomplete, i.e. in the latter case vinyl acetate units remain in the polymer.

The degree of hydrolysis is a criterion of how many groups are converted into hydroxyl groups. The term “polyvinyl alcohol” in connection with a given degree of hydrolysis means therefore, in fact, a vinyl polymer containing ester and hydroxyl groups.

Particularly suitable for the invention are polyvinyl alcohols with the hydrolysis degree between 10% and 99.9%, preferably from 70% to 98%.

Especially preferred di- or polyols are 1,2,3-trihydroxypropane (glycerol), and 2,2-bis(hydroxylmethyl)-1,3-propandiol, 3-propylene glycol and 1,2-propylene glycol.

Preferred acid-components are acid halides of a divalent carboxylic acid and acid halides of multivalent carboxylic acid. Acid halide of a multivalent carboxylic acid is to be understood as carboxylic acid halide with three, four or five acyl halide groups.

Preferred are acid chlorides of a di- or multivalent carboxylic acid. Preference is given to acid halide, such as sebacoyl dichloride, terephthaloyl dichloride, adipoyl dichloride, oxalyl dichloride, succinic acid dichloride, malonic acid dichloride, glutaric acid dichloride, fumaric acid dichloride, tricarballylyl trichloride and 1,2,4,5-benzenecarbonyl tetrachloride, in particular to acid chlorides of dicarboxylic acids such as sebacoyl chloride, terephthaloyl chloride, adipoyl dichloride, oxalyl dichloride and succinic acid dichloride. In one embodiment, the acid chlorides of dicarboxylic acids are selected from sebacoyl chloride, terephthaloyl chloride, and adipoyl dichloride.

Core Material

The core material comprises one or more enzyme. In particular the capsule core material comprises one or more enzymes and water.

According to one preferred embodiment the core material further comprises a di- or polyol. This is the case when the excess of di- or polyol monomer is used to form a polyester shell. Being part of the core material these di- or polyols might have a further beneficial influence. Some of them might act as stabilizers or as anti-microbial agents.

According said preferred embodiment the core material comprises an enzyme, water and a di- or polyol. Suitable di- or polyol are alcohols with two, three, four, five or more hydroxyl groups, especially those which are mentioned above as preferred di- or polyols as starting materials for building the shell.

According this preferred embodiment the core material comprises an enzyme, water and a di- or polyol selected from the group consisting of 1,2,3-trihydroxypropane (glycerol), and 2,2-bis(hydroxylmethyl)-1,3-propandiol, 3-propylene glycol and 1,2-propylene glycol.

Further according this preferred embodiment these preferred polyols are polyols with a degree of polymerization (DP) in the range from 10 to 6000, especially the above mentioned polyvinylalcohols.

Enzymes

As described herein, the enzyme-containing core can comprise one or more enzymes. The enzyme can be, for example, any enzyme capable of degrading polymeric substances, including but not limited to polysaccharides present in filter cakes, fracturing and blocking gel, as well as in other applications/fluids used in the hydrocarbon recovery. For example, the enzyme can be a hydrolase. Non-limiting examples of the enzyme include cellulases, hemicellulases, pectinases, xanthanases, mannanases, galactosidases, glucanases, amylases, amyloglucosidases, invertases, maltases, endoglucanase, cellobiohydrolase, glucosidase, xylanase, xylosidase, arabinofuranosidase, oligomerase, and the like, and any mixtures thereof. The galactosidases can be α-galactosidases, β-galactosidases, or any combination thereof. The glucosidases can be α-glucosidases, β-glucosidases, or any combination thereof. The amylases can be α-amylases, β-amylases, γ-amylases, or any combination thereof. In some embodiments, the enzyme is a thermostable or thermotolerant enzyme.

Preferred are microcapsules wherein the enzyme is a cellulase.

In some embodiments, the enzyme is any of the cellulases derived from hyperthermophilic bacteria and/or non-naturally occurring variants thereof described in PCT publication WO 2009/020459 (the entire disclosure of which is incorporated herein by reference). In some embodiments, the enzyme is encoded by a nucleic acid sequence having at least 70%, 80%, 90%, 95%, 96%, 97%, 98%, 99%, 100%, or a range defined by any two of these values, sequence identity to any of the below-listed DNA sequences described in WO 2009/020459. In some embodiments, the enzyme has an amino acid sequence having at least 70%, 80%, 90%, 95%, 96%, 97%, 98%, 99%, 100%, or a range defined by any two of these values, sequence identity to any of the below-listed protein sequences described in WO 2009/020459. The DNA and protein sequences include: WO 2009/020459 SEQ ID NOS: 1, 2, 3, 6, 7, 8, 9, 10, 11, 12, 13, 14, 15, 16, 17, 18, 19, 20, 21, 22, and 23. In one embodiment the SEQ ID is No. 9.

Besides the above-listed nucleotide and amino acid sequences related to wild-type and evolved variants of the cellulase from Thermotoga maritima strain MSB8, the additional mutants listed in Table 2 and Example 5 (from WO 2009/020459) are also deemed useful as components of the compositions described herein and/or in the methods of making these compositions.

In one preferred embodiments, the enzyme can be a cellulase or a variant of a cellulase disclosed in U.S. Pat. No. 5,962,258, U.S. Pat. No. 6,008,032, U.S. Pat. No. 6,245,547, U.S. Pat. No. 7,807,433, international patent publication WO 2009/020459, international patent publication WO 2013/148163, the contents of which are incorporated by reference in their entireties. In some embodiments, the cellulase can be a commercially available product including, but not limited to, PYROLASE® 160 cellulase, PYROLASE® 200 cellulase, or PYROLASE® HT cellulase (BASF Enzymes LLC, San Diego, Calif.), or any mixture thereof. In some embodiments, the cellulase is PYROLASE® HT cellulase.

Additional Components

In addition to the enzyme, the enzyme-containing core may include one or more additional components. Non-limiting examples of the additional component include stabilizers, buffers, acidifiers and anti-microbial agents. Some component can be multifunctional. For example, in some embodiments, one reagent can have properties to function as a stabilizer, and/or a buffering agent, and/or an acidifying agent, and/or anti-microbial agent, and/or a monomer component for the polymerization reaction of the polyester shell.

In some embodiments, the enzyme-containing core comprises one or more stabilizers. Examples of stabilizers include, but are not limited to, sodium chloride, sodium sulfate, ammonium sulfate, diol (as mentioned above), polyol (as mentioned above) and any combination thereof. The amount of stabilizer in enzyme containing core can be 0%, 1%, 2%, 3%, 4%, 5%, 10%, 15%, 20%, 25%, 30%, 35%, 40%, 45%, 50%, 55%, 60%, 65%, 70%, or any range between these values by weight.

In some embodiments, the enzyme-containing core comprises one or more buffering agents. As used herein, the terms “buffer” and “buffering agent” are used interchangeably, and refer to any substance that can control the pH of the environment in which it is present. Examples of buffers include, but are not limited to, sodium or potassium salt of citrate, sodium or potassium salt of phosphate (monobasic and/or dibasic), succinic acid and its salt, Tris-HCl buffers, morpholino-ethanesulphonic acid (MES) buffers, pyridine, cacodylate buffers, Bis(2-hydroxyethyl)aminotris(hydroxymethyl)methane (BIS-TRIS buffers, piperazine-N,N′-bis(2-ethanesulfonic acid (PIPES) buffers, 3-(N-morpholino)propanesulfonic acid (MOPS) buffers, 3-(N-Morpholino)-2-hydroxypropanesulfonic acid (MOPSO) buffers.

In some embodiments, the enzyme-containing core material comprises one or more-acidifying agents. As used herein, the terms “acidifying agent” and “acidifier” are used interchangeably, and refer to any substance that can lower the pH of the environment in which it is present. For example, the acidifying agent can be an organic acid, or a salt or ester thereof, or an inorganic acid, or a salt or ester thereof.

In some embodiments, the acidifying agent comprises mild acidifying inorganic salts, organic acids, salts of organic acids, polyesters of organic acids, organic buffers, or any combination thereof. Examples of organic buffers include, but are not limited to, Tris-HCl buffers, morpholino-ethanesulphonic acid (MES) buffers, pyridine, cacodylate buffers, Bis(2-hydroxyethyl)-amino-tris(hydroxymethyl)methane (BIS-TRIS buffers, piperazine-N,N′-bis(2-ethanesulfonic acid (PIPES) buffers, 3-(N-morpholino)propanesulfonic acid (MOPS) buffers, 3-(N-Morpholino)-2-hydroxypropanesulfonic acid (MOPSO) buffers, ethylene-diamine-tetraacetic acid (EDTA) buffers, glycine buffers, and any combination thereof. Examples of mild acidifying inorganic salts include, but are not limited to, ammonium sulfate, sodium phosphate monobasic, ammonium chloride, sodium sulfate, potassium phosphate monobasic, magnesium chloride, sodium phosphate dibasic, potassium phosphate dibasic, and any combination thereof. Non-limiting examples of polyesters of organic acid include polylactic acid, poly(lactic-co-glycolic acid), polyglycolic acid, poly(ethylene) therephtalates, polycaprolactone, diphenyl oxalate, and any combination thereof. In some embodiments, the organic acid is citric acid, oxalic acid, malonic acid, glycolic acid, pyruvic acid, lactic acid, maleic acid, aspartic acid, isocitric acid, any salt of these organic acids, or any combination thereof. In some embodiments, the acidifying agent comprises or is an ester, a lactone, polyester, polylactone, or any combination thereof. In some embodiments, the acidifying agent comprises or is an ester. Non-limiting examples of polyester include solid biodegradable polyesters (SBPs), such as polybutylene succinate (PBS), poly(butylene succinate-co-butylene terephthalate) (PBBT), polybutylene terephalate, polyhydroxybutyrate, and any combination thereof.

The amount of acidifying agent in the enzyme-containing core can vary. For example, the amount of the acidifying agent in the enzyme-containing core can be, 0%, 0.1%, 0.2%, 0.5%, 1%, 2%, 5%, 10%, 15%, 20%, 25%, 30%, 35%, 40%, 45%, and up to 50%, of weight, based on the total weight of the microcapsule.

Gel cross linking is often used to increase the viscosity and effectiveness of fracturing fluid. Some cross linking reactions require high pH (e.g. pH 9.5 and above). This alkaline pH of the cross-linked gelled solutions is not ideal for the activity of most enzyme breakers. Without being bound by any particular theory, it is believed that the acidifier present in the formulated enzyme breakers disclosed herein can, in some embodiments, establish a reduced pH environment upon release in which the enzyme can hydrolyze the cross-linked gelled fluid effectively, in particular to a complete break.

In some embodiments, the enzyme-containing core comprises one or more anti-microbial agents. Anti-microbial agent can be a biocide, which kills the microbe, or a preservative, which prevents or limits the growth of microbes without killing the microbes. Examples of anti-microbial agents include, but are not limited to, Proxel® GXL, and glutaraldehyde; benzoic acid, sorbic acid, propionic acid, sulfur oxide, sulfite, metabissulfite, nitrate, nitrite, and their sodium or potassium salts; methyl paraben, ethyl paraben, propyl paraben, heptyl paraben.

Microencapsulation

Preferred are microcapsules which are obtainable by a process comprising the steps:
a) preparation of an emulsion with aqueous disperse phase, a hydrophobic continuous phase and a protective colloid,
   wherein the aqueous disperse phase comprises the core material and at least one alcohol selected from the group consisting of diols and polyols, and
b) subsequent addition of one or more acid halide of a di- or multivalent carboxylic acid
c) and polycondensation of the diol and/or polyol with the acid halide of a di- or multivalent carboxylic acid to build the microcapsule shell.

According to the invention, the core material comprises at least an enzyme.

In the abovementioned preferred process the acid halides of a di- or multivalent carboxylic acid dissolve in the hydrophobic continuous phase. The alcohols are part of the aqueous disperse phase and the polycondensation to build the microcapsule shell predominantly happens at the oil-water interface. Consequently, at least a part of the aqueous phase comprising the core material is enclosed in the microcapsule. Ideally, the entire aqueous phase may be enclosed in the microcapsule but in practice at part of the aqueous phase may not be enclosed. So, the result of the preferred process is a dispersion of microcapsules in the hydrophobic phase, the microcapsules comprising a shell and a core, wherein the shell comprises a polyester and the core comprises—besides the core material—water.

The amount of the diol and/or polyol to be used according to the invention and of the acid halide of a di- or multivalent carboxylic acid varies within the customary scope for interfacial polycondensation processes.

The amount of the acid halide defines the shell thickness. According to one preferred embodiment the diol and/or polyol is used in an excess of the acid halide. Consequently, some diol and/or polyol remains unreacted in the core. Since the polyol has the ability to function as stabilizer and co-solvent of the enzyme, such an excess in relation to the acid halide is preferred.

The halide of the di- or multivalent carboxylic acid are usually used in amounts of from 0.5 to 40% by weight, based on the sum of capsule core material and capsule shell, in particular from 1 to 25% by weight.

Hydrophobic Continuous Phase

The continuous phase consists, to more than 95% by weight a hydrophobic diluent. Herein below, “hydrophobic diluent” means diluents which have a solubility in water of <10 g/l, in particular <5 g/l at 20° C. and atmospheric pressure. In particular, the hydrophobic diluent is selected from
cyclohexane,
glycerol ester oils,
hydrocarbon oils, such as paraffin oil, diisopropylnaphthalene, purcellin oil, perhydrosqualene and solutions of microcrystalline waxes in hydrocarbon oils,
animal or vegetable oils,
mineral oils, the distillation start-point of which under atmospheric pressure is ca. 250° C. and the distillation end-point of which is 410° C., such as e.g. Vaseline oil,
esters of saturated or unsaturated fatty acids, such as alkyl myristates, e.g. isopropyl, butyl or cetyl myristate, hexadecyl stearate, ethyl or isopropyl palmitate and cetyl ricinolate,
silicone oils, such as dimethylpolysiloxane, methyl phenyl polysiloxane and the silicon glycol copolymer,
fatty acids and fatty alcohols or waxes such as carnauba wax, candelilla wax, beeswax, microcrystalline wax, ozokerite wax and Ca, Mg and Al oleates, myristates, linoleates and stearates.

“Glycerol ester oils” means esters of saturated or unsaturated fatty acids with glycerol. Mono-, di- and triglycerides, and their mixtures are suitable. Preference is given to fatty acid triglycerides. Fatty acids which may be mentioned are, for example, C₆-C₁₂-fatty acids such as hexanoic acid, octanoic acid, decanoic acid and dodecanoic acid. Preferred glycerol ester oils are C₆-C₁₂-fatty acid triglycerides, in particular octanoic acid and decanoic acid triglycerides, and their mixtures. Such an octanoyl glyceride/decanoyl glyceride mixture is for example Miglyol® 812 from Hüls or Myritol® 318 from BASF.

Particularly preferred hydrophobic diluents are low-boiling alkanes or alkane mixtures such as cyclohexane, naphtha, petroleum, C₁₀-C₁₂-isoalkanes, as are commercially available as Isopar™ G. Furthermore, particular preference is given to using diisopropylnaphthalene, which is available for example as KMC oil from RKS.

According to the preferred process mentioned above an emulsion with aqueous disperse phase which comprises the core material and a diol and/or polyol, a hydrophobic continuous phase and a protective colloid is built in a first step. In order to obtain a stable emulsion, surface-active substances such as protective colloids are generally required. As a rule, the microcapsules are prepared in the presence of at least one organic protective colloid. These protective colloids may be ionic or neutral. Protective colloids can be used here either individually or else in mixtures of two or more identically or differently charged protective colloids.

The stabilized droplets of the emulsion here have a size which corresponds approximately to the size of the later microcapsules. The shell formation takes place by polycondensation reaction of the monomers, which is started with the addition of the acid halide.

Protective Colloids

In order to obtain a stable emulsion and a homogeneous shell formation, a protective colloid is used. In particular the protective colloid is an amphiphilic polymer. According to one embodiment the amphiphilic polymer is obtained by free-radical polymerization of a monomer composition comprising ethylenically unsaturated hydrophilic monomers II and ethylenically unsaturated hydrophobic monomers I. The amphiphilic polymer here especially exhibits a statistical distribution of the monomer units.

The amphiphilic polymer is in particular positioned, on account of its monomer composition comprising both hydrophilic and hydrophobic units, at the interface of the emulsion droplets and stabilizes these.

Suitable ethylenically unsaturated hydrophobic monomers I comprise long-chain monomers with C₈-C₂₀-alkyl radicals. Of suitability are, for example, alkyl esters of C₈-C₂₀-alcohols, in particular C₁₂- to C₂₀-alcohols, in particular C₁₆-C₂₀-alcohols, with ethylenically unsaturated carboxylic acids, in particular with ethylenically unsaturated C₃-C₆-carboxylic acids such as acrylic acid, methacrylic acid, fumaric acid, itaconic acid and aconitic acid. By way of example, mention may be made of dodecyl acrylate, dodecyl methacrylate, tridecyl acrylate, tridecyl methacrylate, tetradecyl acrylate, tetradecyl methacrylate, octadecyl acrylate, octadecyl methacrylate. Particular preference is given to octadecyl acrylate and octadecyl methacrylate.

Within the context of the ethylenically unsaturated hydrophilic monomers II, hydrophilic means that they have a solubility in water of >50 g/l at 20° C. and atmospheric pressure.

Suitable ethylenically unsaturated hydrophilic monomers II are ethylenically unsaturated monomers with acid groups, and salts thereof, ethylenically unsaturated quaternary compounds, hydroxy (C₁-C₄)alkyl esters of ethylenically unsaturated acids, alkylaminoalkyl (meth)acrylates and alkylaminoalkyl(meth)acrylamides. Ethylenically unsaturated hydrophilic monomers with acid groups or salts of acid groups that may be mentioned by way of example are acrylic acid, methacrylic acid, 2-acrylamide-2-methylpropanesulfonic acid, itaconic acid, maleic acid, fumaric acid. Ethylenically unsaturated quaternary compounds that may be mentioned are dimethylaminoethyl acrylate or methacrylates which are quaternized with methyl chloride. Further suitable ethylenically unsaturated hydrophilic monomers are maleic anhydride and acrylamide.

Besides the ethylenically unsaturated hydrophobic monomers (monomers I) and the ethylenically unsaturated hydrophilic monomers (monomers II), the amphiphilic polymer can also comprise further comonomers (monomers III) in polymerized-in form which are different from the monomers of groups I and II. Ethylenically unsaturated comonomers of this type can be chosen to modify the solubility of the amphiphilic polymer.

Suitable other monomers (monomers III) are nonionic monomers which optionally have C₁-C₄-alkyl radicals. In particular, the other monomers are selected from styrene, C₁-C₄-alkylstyrenes such as methylstyrene, vinyl esters of C₃-C₆-carboxylic acids such as vinyl acetate, vinyl halides, acrylonitrile, methacrylonitrile, ethylene, butylene, butadiene and other olefins, C₁-C₄-alkyl esters and glycidyl esters of ethylenically unsaturated carboxylic acids. Preference is given to C₁-C₄-alkyl esters and glycidyl esters of ethylenically unsaturated C₃-C₆-carboxylic acids such as acrylic acid, methacrylic acid, fumaric acid, itaconic acid and aconitic acid, for example methyl acrylate, methyl methacrylate, butyl acrylate or butyl methacrylate, and glycidyl methacrylate.

The weight ratio of ethylenically unsaturated hydrophobic monomers/ethylenically unsaturated hydrophilic monomers is in particular 95/5 to 20/80, especially 90/10 to 30/60.

The amphiphilic polymers comprise in a preferred form at least 20% by weight, particularly at least 30% by weight, in particular 40% by weight and especially at least 45% by weight, and in particular at most 95% by weight, especially at most 90% by weight, of ethylenically unsaturated hydrophobic monomers I in polymerized-in form, based on the total weight of the monomers.

The amphiphilic polymers comprise in a preferred form at least 5% by weight, in particular at least 7% by weight, and especially at least 10% by weight, and in a preferred form at most 80% by weight, in particular at most 60% by weight, and especially at most 50% by weight, of ethylenically unsaturated hydrophilic monomers II in polymerized-in form, based on the total weight of the monomers.

The amphiphilic polymers comprise in a preferred form at least 5% by weight, in particular at least 7% by weight, in particular 10% by weight, and in a preferred form at most 55% by weight, in particular at most 45% by weight, of other monomers III in polymerized-in form, based on the total weight of the monomers.

Preference is given to using amphiphilic polymers which are obtainable by free-radical polymerization of a monomer composition comprising, in particular consisting of

| | |
|---|---|
| 20 to 90% by weight | of one or more ethylenically unsaturated |
| | hydrophobic monomers (monomers I), |
| 5 to 50% by weight | of one or more ethylenically unsaturated |
| | hydrophilic monomers (monomers II), |
| 0 to 45% by weight | of one or more other monomers (monomers III) |

in each case based on the total weight of the monomers I, II and III.

Particular preference is given to choosing amphiphilic polymers which are obtainable by free-radical polymerization of a monomer composition comprising, in particular consisting of

| | |
|---|---|
| 20 to 90% by weight | of one or more alkyl esters of C₈-C₂₀-alcohols with ethylenically |
| | unsaturated carboxylic acids, |
| 5 to 50% by weight | of one or more monomers selected from ethylenically unsaturated |
| | monomers with acid groups, and salts thereof, ethylenically unsaturated |
| | quaternary compounds, hydroxy (C₁-C₄)alkyl esters of ethylenically |
| | unsaturated acids, alkylaminoalkyl (meth)acrylates, alkylaminoalkyl |
| | (meth)acrylamides, maleic anhydride and acrylamide, |
| 0 to 45% by weight | of one or more monomers selected from styrene, C₁-C₄-alkylstyrenes, |
| | vinyl esters of C₃-C₆-carboxylic acids, vinyl halides, acrylonitrile, |
| | methacrylonitrile, ethylene, butylenes, butadiene and C₁-C₄-alkyl esters |
| | of ethylenically unsaturated C₃-C₆-carboxylic acids |

in each case based on the total weight of the monomers.

Particular preference is given to amphiphilic polymers which are obtainable by free-radical polymerization of a monomer composition comprising, in particular consisting of,

| | |
|---|---|
| 40 to 90% by weight | of one or more alkyl esters of C₁₆-C₂₀-alcohols with ethylenically |
| | unsaturated carboxylic acids, |
| 10 to 35% by weight | of one or more monomers selected from acrylic acid, methacrylic acid, |
| | 2-acrylamido-2-methylpropanesulfonic acid, itaconic acid, maleic acid, |
| | fumaric acid, maleic anhydride and acrylamide, |
| 0 to 40% by weight | of one or more monomers selected from styrene, C₁-C₄-alkylstyrenes, |
| | vinyl esters of C₃-C₆-carboxylic acids, vinyl halides, acrylonitrile, |
| | methacrylonitrile and methyl methacrylate |

in each case based on the total weight of the monomers.

Furthermore, preference is given to amphiphilic polymers which are obtainable by free-radical polymerization of a monomer composition comprising, in particular consisting of,

| | |
|---|---|
| 60 to 90% by weight | of one or more alkyl esters of C₁₆-C₂₀-alcohols with ethylenically |
| | unsaturated carboxylic acids, |
| 10 to 35% by weight | of one or more monomers selected from acrylic acid, methacrylic acid, |
| | 2-acrylamido-2-methylpropanesulfonic acid, itaconic acid, maleic acid, |
| | fumaric acid, maleic anhydride and acrylamide, |
| 0 to 10% by weight | of one or more monomers selected from styrene, C₁-C₄-alkylstyrenes, |
| | vinyl esters of C₃-C₆-carboxylic acids, vinyl halides, acrylonitrile, |
| | methacrylonitrile and methyl methacrylate |

in each case based on the total weight of the monomers.

Furthermore, preference is given to amphiphilic polymers which are obtainable by free-radical polymerization of a monomer composition comprising, in particular consisting of,

| | |
|---|---|
| 40 to 70% by weight | of one or more alkyl esters of C₁₆-C₂₀-alcohols with ethylenically |
| | unsaturated carboxylic acids, |
| 10 to 35% by weight | of one or more monomers selected from acrylic acid, methacrylic acid, |
| | 2-acrylamido-2-methylpropanesulfonic acid, itaconic acid, maleic acid, |
| | fumaric acid, maleic anhydride and acrylamide, |
| 10 to 40% by weight | of one or more monomers selected from styrene, C₁-C₄-alkylstyrenes, |
| | vinyl esters of C₃-C₆-carboxylic acids, vinyl halides, acrylonitrile, |
| | methacrylonitrile and methyl methacrylate |

in each case based on the total weight of the monomers.

The amphiphilic polymer generally has an average molecular weight M_{w} (determined by means of gel permeation chromatography) of from 5000 to 500 000, in particular from ≥10 000 up to 400 000 and particularly in particular 30 000 to 200 000.

The amphiphilic polymers are in particular prepared by initially introducing the total amount of the monomers in the form of a mixture and then carrying out the polymerization. Furthermore, it is possible to meter in the monomers under polymerization conditions discontinuously in one or more part amounts or continuously in constant or changing quantitative streams.

The optimum amount of amphiphilic polymer for stabilizing the hydrophilic droplets before the reaction and the microcapsules after the reaction is influenced firstly by the amphiphilic polymer itself, secondly by the reaction temperature, the desired microcapsule size and by the shell materials, and also the core composition. The optimally required amount can be ascertained easily by persons of ordinary skill in the art. As a rule, the amphiphilic polymer is used for preparing the emulsion in an amount of from 0.01 to 15% by weight, in particular 0.05 to 12% by weight and especially 0.1 to 10% by weight, based on the capsules (shell and core).

Process of Polymerization

According to the preferred process mentioned above an emulsion with aqueous disperse phase which comprises the core material and a diol and/or polyol, a hydrophobic continuous phase and a protective colloid is built in a first step.

According to one preferred embodiment the protective colloid is added as part of the oil phase.

The emulsion is made by mixing the components, i.e. by vigorously stirring the components of the emulsion or by using suitable dispersing device/homogenizing devices. In one embodiment, a first premix of the components of the aqueous disperse phase and separately a second premix containing the hydrophobic solvent and the protective colloid. Thereafter, the first premix and the second premix are mixed in order to obtain a water-in-oil emulsion. Thereafter, the acid halide of di- and/or polycarboxylc acids optionally mixed with a hydrophobic solvent is added. The acid halide of di- and/or polycarboxylc acids dissolves in the hydrophobic continuous phase and polymerization predominantly takes place at the interface between the hydrophobic and the hydrophilic phase thereby forming the shell.

The capsule size can be controlled within certain limits via the rotational speed of the dispersing device/homogenizing device and/or with the help of the concentration of the amphiphilic polymer and/or via its molecular weight, i.e. via the viscosity of the continuous phase. As a rule, the size of the dispersed droplets decreases as the rotational speed increases up to a limiting rotational speed.

In this connection, it is important that the dispersing devices are used at the start of capsule formation. For continuously operating devices with forced throughflow, it is sometimes advantageous to pass the emulsion through the shear field several times.

As a rule, the polymerization is carried out at 20 to 85° C., in particular at room temperature. Expediently, the polymerization is performed at atmospheric pressure, although it is also possible to work at reduced or slightly increased pressure.

The reaction time of the polycondensation is normally 1 to 10 hours, mostly 2 to 5 hours.

After polymerization according to the process mentioned above a dispersion of the microcapsules in the hydrophobic phase is obtained. In particular dispersions with a content of from 5 to 50% by weight of microcapsules, can be produced by the process according to the invention. The microcapsules are individual capsules. Correspondingly a dispersion comprising 5 to 50% by weight, based on the total weight of the dispersion, of microcapsules in a hydrophobic solvent has been found.

The microcapsules obtained can be isolated by removing the hydrophobic solvent. This can be performed for example by filtration centrifugation or evaporating off the hydrophobic solvent or by means of suitable spray-drying processes.

For better handling the particles may be further processed, e.g. by agglomeration of fine powders of microcapsules to larger particles, of course without modifying the primary particle size of 0.5 μm to 20 μm, e.g. by granulating or pelletizing. For this purpose, optionally inorganic or organic binders may be used as additives. Examples of such binders include silicates. In other embodiments, the material may be packed into bags of a water soluble material.

It may also be possible to redisperse the microcapsules in water, thereby obtaining an aqueous dispersion of the microcapsules according to the present invention.

Use of the Microcapsules in Oilfield Applications

The microcapsules as described herein may be used for various oilfield applications such as the treatment of filter cakes or reducing the viscosity of fluids used in oilfield applications, including but not limited to fluids for the treatment of subterranean formations.

As disclosed herein, microcapsules with a core-material comprising enzymes capable of reducing viscosity of one or more fluids used in hydrocarbon recovery can be formed so that the enzyme can be protected chemically and/or physically from conditions prevailing in subterranean formations which may adversely influence the performance of the enzymes, such as for example unsuitable temperature, pressure, or pH conditions. Furthermore, the microcapsules may advantageously delay the onset of the effect of the enzymes.

For example, microencapsulated enzyme disclosed herein can be added to any subterranean treatment fluid, in particular subterranean treatment fluids comprising polymers, in particular viscosifying polymers known in the art to reduce its viscosity. Suitable examples of subterranean treatment fluids include, but are not limited to, drilling fluids, fracturing fluids, carrier fluids, diverting fluids, gravel packing fluids, completion fluids, workover fluids, and the like in downhole conditions.

Correspondingly, a method of treating a subterranean formation has been found, comprising contacting a subterranean formation with an aqueous treatment fluid, wherein the treatment fluid comprises the microcapsules according to the invention.

Correspondingly, in one embodiment of the invention an aqueous fracturing fluid has been found, wherein the aqueous fracturing fluid comprises at least
A) an aqueous base fluid,
B) a proppant,
C) a viscosifier, and
D) microcapsules according to the invention.

Correspondingly, in a further embodiment of the invention a method of fracturing a subterranean formation has been found, which at least comprises the steps of
(1) formulating an aqueous fracturing fluid,
(2) pumping the fracturing fluid down the wellbore at a rate and pressure sufficient to flow into the formation and to initiate or extend fractures in the formation,
(3) reducing the applied pressure thereby allowing at least a portion of the injected fracturing fluid to flow back from the formation into the wellbore, and
(4) removing such flowed back fracturing fluid from the wellbore,
   wherein the aqueous fracturing fluid comprises at least
A) an aqueous base fluid,
B) a proppant,
C) a viscosifier, and
D) microcapsules according to the invention.

In other embodiments, additional compositions may be included into the fracturing fluid, such as for example flowback aids (e.g., a surfactant, solvent, or a cosolvent). Such flowback aids assist in removing capillary pressure or surface tension, allowing the injected fracturing fluid to flow from the formation after a hydraulic fracturing treatment.

A) Aqueous Base Fluid

The aqueous base fluid for the fracturing fluid comprises water.

Besides water the aqueous formulation may also comprise organic solvents miscible with water. Examples of such solvents comprise alcohols such as ethanol, n-propanol, i-propanol or butyl diglycol. If organic solvents are present their amount should not exceed 50% by weight with respect to the solvents present in the aqueous base fluid. In a preferred embodiment of the invention the aqueous base fluid comprises at least 70% by weight of water with respect to the solvents present in the aqueous base fluid, more in particular at least 90% by weight. In a further preferred embodiment of the invention only water is used as solvent in the aqueous base fluid.

The aqueous base fluid may comprise dissolved salts. Examples of salts comprise halogenides, in particular chlorides, sulfates, borates of mono- or divalent cations such as Li⁺, Na⁺, K⁺, Mg²⁺, Ca²⁺, Sr²⁺, or Ba²⁺. In a one embodiment of the invention, the aqueous fracturing fluid comprises at least one salt.

In particular, the salt may be KCl and/or ammonium chloride. The salinity of the water, in particular the concentration of KCl and/or ammonium chloride may be from 0.1% by weight to 10% by weight relating to the aqueous base fluid, in particular from 0.5% to 8% by weight, especially from 1% to 6% by weight and by the way of example 3 to 5% by weight.

B) Proppants

The aqueous fracturing fluid furthermore comprises at least one proppant which is suspended in the aqueous fracturing fluid. Proppants are small hard particles which prevent the fractures from closing after formation of the fractures and subsequent removal of pressure. Suitable proppants are known to the skilled artisan. Examples of proppants include naturally-occurring sand grains, resin-coated sand, sintered bauxite, ceramic materials, glass materials, polymer materials, ultra lightweight polymer beads polytetrafluoroethylene materials, nut shell pieces, cured resinous particulates comprising nut shell pieces, seed shell pieces, cured resinous particulates comprising seed shell pieces, fruit pit pieces, cured resinous particulates comprising fruit pit pieces, wood, composite particulates, and any combinations thereof.

The amount of proppants in the aqueous fracturing fluid may be from 50 kg/m³ to 3500 kg/m³ of the fracturing fluid, in particular from 50 kg/m³ to 1200 kg/m³ of the fracturing fluid.

C) Viscosifiers

The aqueous fracturing fluid furthermore comprises at least one viscosifier for increasing the viscosity of the fracturing fluid. The viscosifier facilitates suspension of the proppant(s) in the aqueous fracturing fluids and reduces its tendency to sediment during delivery into the formation.

Suitable viscosifiers for fracturing fluids are known to the skilled artisan. Viscosifying agents may be water-soluble, thickening polymers or low molecular components such as viscosifying surfactants with glycosidic bonds or combinations thereof.

Non-limiting examples of thickening polymers for use as viscosifier include hydroxyethylcellulose, carboxymethyl cellulose, hydroxyalkyl guar, hydroxyalkyl cellulose, carboxyalkylhydroxy guar, carboxyalkylhydroxyalkyl guar, starch, gelatin, poly(vinyl alcohol), poly(ethylene imine), guar gum, xanthan gum, polysaccharide, cellulose, synthetic polymers, any derivatives thereof, and any combinations thereof. In some embodiments, the viscosifier is present in the aqueous fracturing fluid in a concentration from about 1.8 kg/m³ to about 9.6 kg/m³.

In some embodiments, the viscosifier comprises one or more hydratable polymers. The hydratable polymers can be underivatized guars, derivatized guars, or any combination thereof. It can be advantageous in some embodiments to use underivatized guar. Examples of derivatized guars include, but are not limited to, hydroxypropyl guar and carboxymethyl hydroxypropyl guar.

In one preferred embodiment the viscosifier comprises at least one polysaccharide and/or polysaccharide derivative. Of course also a mixture of two or more polysaccharides and/or polysaccharide derivatives may be used. The polysaccharides and/or polysaccharide derivatives are water-soluble and act as thickeners for the aqueous fracturing fluid. The thickening effect may be enhanced by the use of the crosslinkers.

Examples of suitable polysaccharide and/or polysaccharide derivatives comprise xanthans, scleroglucanes, galactomannan gums or cellulose derivatives.

Galactomannan gums comprise a backbone of mannose units with various amounts of galactose units attached thereto. In certain embodiments, the ratio of mannose/galactose may be from 1.6 to 2, for example from 1.6 to 1.8. The galactose units may be distributed regularly or randomly along the backbone. In certain embodiments, the average molecular weight M_{w} may be from 1,000,000 g/mol to 2,000,000 g/mol.

In one embodiment, the polysaccharides and/or polysaccharide derivatives are galactomannan gums and/or galactomannan gum derivatives. Examples of suitable galactomannan gums include gum arabic, gum ghatti, gum karaya, tamarind gum, tragacanth gum, guar gum, or locust bean gum. Examples of derivatives include hydroxyethylguar, hydroxypropylguar, carboxymethylguar, carboxymethyl hydroxyethylguar and carboxymethyl hydroxypropylguar.

Examples of suitable cellulose derivatives include hydroxyethyl cellulose, carboxyethylcellulose, carboxymethylcellulose, or carboxymethylhydroxyethylcellulose.

In one embodiment of the invention, the polysaccharide and/or polysaccharide derivative is guar gum and/or a guar gum derivative. In a preferred embodiment, the polysaccharide and/or polysaccharide derivative is carboxymethyl hydroxypropyl guar.

In certain embodiments the amount of carboxymethyl groups in carboxymethyl hydroxypropyl guar expressed as degree of substitution (DS), i.e. the average number of OH-groups per sugar molecule substituted, may be from 0.1 to 0.2.

In certain embodiments the amount of hydroxypropyl groups in carboxymethyl hydroxypropyl guar expressed as molar substitution (MS), i.e. the average number of propylene oxide groups per sugar molecule, may be from 0.2 to 0.3.

The amount of polysaccharides and/or polysaccharide derivatives (B) may be from 0.05% to 2% by weight, relating to the base fluid. Preferably, the amount is from 0.1 to 1.5% by weight and more preferably from 0.2 to 1.0% by weight.

Aqueous fracturing fluids according to the invention comprising polymers as viscosifiers, in particular aqueous fracturing fluids comprising polysaccharides and/or polysaccharide derivatives may comprise in addition cross-linking agents. Crosslinking agents may be used by the skilled artisan to additionally increase the viscosity of the fracturing fluid.

Examples of crosslinking agents include, but are not limited to, borate ions, zirconate ions, titanate ions, metal ions such as aluminum-, antimony-, zirconium-, and titanium-containing compounds and any combination thereof. Also certain organo-metallic compounds may be used such as organotitanates.

In a preferred embodiment the crosslinking agent comprises boron compounds such as borates or boron releasing compounds. Non-limiting examples of borate cross-linkers include organoborates, monoborates, polyborates, mineral borates, boric acid, sodium borate, including anhydrous or any hydrate, borate ores (e.g., colemanite or ulexite), and any other borate complexed to organic compounds to delay the release of the borate ion. In some embodiments, cross-linking agents can be in the forms of instant cross-linkers; in some other embodiments, the cross-linking agents can be in the forms of delayed cross-linkers based on delayed release formulations.

Such boron comprising crosslinkers are preferably used for crosslinking polysaccharides and/or polysaccharide derivatives, in particular for crosslinking galactomannan gums, preferably guar gums and/or a guar gum derivatives. In a preferred embodiment, the polysaccharide and/or polysaccharide derivative to be crosslinked is carboxymethyl hydroxypropyl guar.

D) Microcapsules as Described Above

The aqueous fracturing fluid according to the present invention furthermore comprises microcapsules according to the present invention. Of course also mixtures of different microcapsules according to the present invention may be used.

Without being bound by any particular theory, it is believed that the microcapsule shells disclosed herein can function, as protective coatings for the enzyme-containing core prior to the enzyme release from the harsh high temperature, and sometimes high pH environment of fracturing fluids in subterranean formation.

In some embodiments, the enzyme is cellulase. In some embodiments, the enzyme is mannanase. Such enzymes are in particular useful in embodiments using guar polymers as viscosifiers. The cellulase or mannanase may hydrolyze the guar polymer at temperatures in excess of 70° C. as well as in excess of 80° C. In fact, the cellulase may hydrolyze the guar polymer at temperatures in excess of 85° C. and even in excess of 90° C. In addition, the cellulase or mannanase may be used in combination with other enzymes and/or oxidative breakers to degrade guar gels over broader temperature and pH ranges. The microcapsules disclosed herein can be used to break, for example subterranean treatment fluids, at relatively high temperature ranges, e.g. at temperatures from 65° C. to 125° C.

The pH of the fluids can also vary. For example, the pH of the fluid can be, or be about, 5.0 to 13.0. In some embodiments, the pH of the fluid is, or is about, 5.0, 5.5, 6.0, 6.5, 7.0, 7.5, 8.0, 8.5, 9.0, 9.5, 10.0, 10.5, 11.0, 11.5, 12.0, 12.5, 13.0, or a range between any two of these values. Alkaline pH (e.g., a pH of 9.5 and above) is often required for cross-linked gel solution to increase the viscosity and therefore effectiveness of the fracturing fluid. In one embodiment, the pH is from 9.5 to 12.0.

Further Components

Besides the components above, the aqueous fracturing fluid may optionally comprise further components. Examples of such further components comprise bases, biocides, buffers, clay stabilizers, corrosion inhibitors, defoamers, non-emulsifying agents, flowback aids, scale inhibitors, oxygen scavengers, friction reducers, breakers or surfactants.

Preferred Aqueous Fracturing Fluids

In a preferred embodiment of the invention, the aqueous fracturing fluid comprises at least
A) an aqueous base fluid,
B) a proppant,
C) a polymeric viscosifier, which comprises at least one polysaccharide and/or polysaccharide derivative and
D) microcapsules according to the invention, wherein the enzyme is a cellulase.

Preferred polymers C) and microcapsules D) have been disclosed above.

In a another preferred embodiment of the invention, the aqueous fracturing fluid comprises at least
A) an aqueous base fluid,
B) a proppant,
C) a polymeric viscosifier, which comprises at least one galactomannan gums and/or galactomannan gum derivatives and
D) microcapsules according to the invention, wherein the enzyme is a cellulase.

Preferred polymers C) and microcapsules D) have been disclosed above.

In a another preferred embodiment of the invention, the aqueous fracturing fluid comprises at least
A) an aqueous base fluid,
B) a proppant,
C) a polymeric viscosifier, which comprises at least one is guar gum and/or a guar gum derivative and a crosslinker, preferably a boron containing crosslinker, and
D) microcapsules according to the invention, wherein the enzyme is a cellulase,
   and wherein the pH value of the aqueous fracturing fluid is rom 9.5 to 12.0.

Method of Fracturing

The method of fracturing a subterranean formation according to the present invention may be applied to any subterranean formation, preferably hydrocarbon containing subterranean formations. The hydrocarbons may be oil and/or gas. Besides oil and/or gas the formations may contain water which usually comprises salts. The salinity of the formation water may be for instance from 10,000 ppm to 230,000 ppm.

The formations may be sandstone, carbonate or shale formations. The formation temperature usually is above room temperature and may be at least 35° C., preferably at least 40° C., more preferably at least 50° C. The formation temperature may be up to 175° C. Preferably, the formation temperature may be from 60° C. to 130° C., more preferably from 65° C. to 125° C., and for example from 70° C. to 90° C.

For applying the method according to the present invention to the formation, the formation is penetrated by at least one wellbore. The wellbore may be a “fresh” wellbore drilled into the formation which needs to become prepared for oil and/or gas production. In another embodiment the wellbore may be a production well which already has been used for producing oil and/or gas but the production rate decreased and it is necessary to fracture the formation (again) in order to increase production.

In the method of fracturing a subterranean formation according to the present invention the aqueous fracturing fluid is injected into at least one wellbore at a rate and pressure sufficient to flow into the formation and to initiate or extend a fracture in the formation. In order to initiate or to extend fractures in the formation a bottomhole pressure sufficient to open a fracture in the formation is necessary. The bottomhole pressure is determined by the surface pressure produced by the surface pumping equipment and the hydrostatic pressure of the fluid column in the wellbore, less any pressure loss caused by friction. The minimum bottomhole pressure required to initiate and/or to extend fractures is determined by formation properties and therefore will vary from application to application. Methods and equipment for fracturing procedures are known to the skilled artisan. The aqueous fracturing fluid simultaneously transports suspended proppants and the proppant becomes deposited into the fractures and holds fractures open after the pressure exerted on the fracturing fluid has been released.

If a crosslinker is present it increases the viscosity of the aqueous fracturing fluid. At high degrees of crosslinking a highly viscous polymer gel may be formed. An increased viscosity aids in properly distributing the proppant (B) in the fluid. In fluids having a low viscosity, the proppants may sediment. The rate of crosslinking increases with increasing temperatures.

When flowing through the wellbore and penetrating into the subterranean formation the aqueous fracturing fluid warms up depending upon the formation temperature thereby increasing the rate of crosslinking. So, crosslinking mainly happens after the aqueous fracturing fluid has been injected into the wellbore. While handling at the surface, the aqueous fracturing fluid has a relatively low viscosity while the fluid in the formation has a higher viscosity.

In one embodiment, the temperature of the aqueous fracturing fluid before injection into the formation is less than 35° C., preferably less than 30° C., more preferably 15° C. to 25° C. Formation temperatures have already been mentioned above.

Furthermore, increasing the temperature has the effect that to microcapsules start to release the enzyme and optionally other components comprised in the core. Consequently, the enzyme may impact on the viscosifier thereby decreasing the viscosity of the viscosifier, e.g. by (partial) depolymerisation of polymeric viscosifiers.

The eventual loss of integrity of the shell material and contact of the enzyme with the viscosified subterranean treatment fluid may occur through one or more mechanisms including, but not limited to, fragmentation of the encapsulating shell, for example through the thermal hydrolysis of the ester linkage, direct dissolution (e.g., direct dissolution of the enzyme into the surrounding fluid through incomplete encapsulating shell coverage), diffusion (e.g., diffusion of the enzyme molecules through the pores of the shell into the surrounding fluid), and the like. In particular, the thermal hydrolysis of the polyester shell provides a unique release mechanism because of the high temperature environment for deep subterranean formation.

The microcapsules comprising the enzyme according the present invention furthermore exhibit a delayed enzyme release pattern. In some embodiments, it can take a time period that is from 20 to 360 minutes in particular from 30 to 180 minutes, to release 20-100% of the enzyme present in the microcapsules to the target composition (e.g., fracturing fluids, drilling fluids, completion fluids, workover fluids, gravel packing fluids, and any combination thereof) from the time that the microcapsules come in contact with the target composition. Persons of ordinary skill in the art will be able to determine the desired delay time according to factors including but not limited to, desired use, well conditions (e.g., depth, temperature, pressure, and any combination thereof), composition of the target composition, and a combination thereof.

The alkaline pH of the cross-linked gelled solutions (e.g. a pH 9.5 and above) is not ideal for the activity of most enzyme breakers. The thermal hydrolysis of polyester shell into acids and optional acidifier present in the enzyme-containing core disclosed herein can, in some embodiments, establish a reduced pH environment upon release in which the enzyme can hydrolyze the cross-linked gelled fluid effectively, in particular to a complete break.

After creating new fractures or extending existing fractures in the formation, the applied pressure is reduced thereby allowing the fractures to close. Proppant (B) “props” fractures open and fracturing fluid is shut in or allowed to flow back. Typically, at least a part of the fluid injected may flow back to the wellbore. The aqueous fracturing fluid flown back from the formation into the wellbore is removed from the wellbore.

It goes without saying for the skilled artisan that the fluid recovered may no longer have exactly the same composition than the injected fracturing fluid: Proppants (B) injected remain in the formation and the injected fluid may be mixed with formation fluids such as oil and/or formation water. Furthermore, polymeric visciosifiers such as guar gum or derivatives thereof have been depolymerized at least to a certain extent thereby decreasing the viscosity of the fluid. The total amount of fluid recovered usually depends on the formation, for instance on how much water the formation adbsorbs and absorbs into its structure. Additionally, fluid may be lost to the formation.

In a preferred embodiment of the invention the method of fracturing a subterranean formation comprises at least the steps of
(1) formulating an aqueous fracturing fluid,
(2) pumping the fracturing fluid down the wellbore at a rate and pressure sufficient to flow into the formation and to initiate or extend fractures in the formation,
(3) reducing the applied pressure thereby allowing at least a portion of the injected fracturing fluid to flow back from the formation into the wellbore, and
(4) removing such flowed back fracturing fluid from the wellbore,
   wherein the aqueous fracturing fluid comprises at least
A) an aqueous base fluid,
B) a proppant,
C) a polymeric viscosifier, which comprises at least one polysaccharide and/or polysaccharide derivative and
D) microcapsules according to the invention, wherein the enzyme is a cellulase,
and wherein the formation temperature is from 60° C. to 130° C., preferably from 65° C. to 125° C., and more preferably from 70° C. to 90° C.

In another preferred embodiment of the invention the method of fracturing a subterranean formation comprises at least the steps of
(1) formulating an aqueous fracturing fluid,
(2) pumping the fracturing fluid down the wellbore at a rate and pressure sufficient to flow into the formation and to initiate or extend fractures in the formation,
(3) reducing the applied pressure thereby allowing at least a portion of the injected fracturing fluid to flow back from the formation into the wellbore, and
(4) removing such flowed back fracturing fluid from the wellbore,
   wherein the aqueous fracturing fluid comprises at least
A) an aqueous base fluid,
B) a proppant,
C) a polymeric viscosifier, which comprises at least one galactomannan gums and/or galactomannan gum derivatives and
D) microcapsules according to the invention, wherein the enzyme is a cellulase,
and wherein the formation temperature is from 60° C. to 130° C., preferably from 65° C. to 125° C., and more preferably from 70° C. to 90° C.

In another preferred embodiment of the invention the method of fracturing a subterranean formation comprises at least the steps of
(1) formulating an aqueous fracturing fluid,
(2) pumping the fracturing fluid down the wellbore at a rate and pressure sufficient to flow into the formation and to initiate or extend fractures in the formation,
(3) reducing the applied pressure thereby allowing at least a portion of the injected fracturing fluid to flow back from the formation into the wellbore, and
(4) removing such flowed back fracturing fluid from the wellbore,
   wherein the aqueous fracturing fluid comprises at least
A) an aqueous base fluid,
B) a proppant,
C) a polymeric viscosifier, which comprises at least one is guar gum and/or a guar gum derivative and a crosslinker, preferably a boron containing crosslinker, and
D) microcapsules according to the invention, wherein the enzyme is a cellulase,
   wherein the pH value of the aqueous fracturing fluid is rom 9.5 to 12.0, and
   wherein the formation temperature is from 60° C. to 130° C., preferably from 65° C. to 125° C., and more preferably from 70° C. to 90° C.

### EXAMPLES

Additional embodiments are disclosed in further detail in the following examples, which are not in any way intended to limit the scope of the claims.

The microcapsules size (arithmetic mean, sum of all sizes divided by the number of particles) was determined by optical microscopy (Leica DM 5000 B) and diameter measurements from 3 batches (in each batch 100 capsules were measured). Diameter measurements were conducted with software for scientific image analysis (Leica Application Suite V 3.8).

D50 means that 50% of the particles have a particle size less than/equal to this value.

Amphiphilic polymer solution 51 was used as the protective colloid: polymer of 88 equivalents by weight stearyl methacrylate and 12 equivalents by weight methacrylic acid, in the form of a 31.0% strength by weight solution in Isopar™ G.

Polyester capsules were prepared as follows:

### Example 1

Preparation of Premixes:
Premix 1:—76 g of demineralized water,
   76 g of an aqueous cellulase enzyme solution (pH 6) containing
      2% by weight of a cellulase for high-temperature applications (Seq. ID No. 2 of WO 2013/148163 A1)
      35% by weight of 1,2,3-trihydroxypropane (glycerol),
      20 mMol sodium citrate,
      0.15% by weight of a biocide (1,2-benzisothiazolin-3-one, 20% by wt. solution in water and dipropylene glycol, Proxel® GXL) and
   9.72 g of 25% aq. sodium hydroxide solution
Premix 2: A premix of 186.92 g of isoparaffin having an initial boiling point of ˜161° C. (Isopar® G, ExxonMobil) oil and 21.64 g of amphiphilic polymer solution S1 was prepared.
Premix 3: A premix of 6.16 g of terephthaloyl chloride (TPC) and 55.28 g dibutyl adipate (dibutyl adipate serves as solvent) was prepared.

Synthesis:

Premix 1 and 2 was transferred in a reactor and emulsified using a high shear homogenizer at the speed equal to 8000 rpm for about 5 minutes, obtaining a water/oil emulsion. Afterwards, at 5000 rpm premix 3 was added at once.

Then, under stirring by means of a blade stirrer at 200 rpm, mixture was heated up to 70° C. and kept at this temperature for 1 hour. Finally, the suspension of the capsules in Isopar® was cooled down to room temperature.

Capsules size: d50=2.9 μm

### Example 2

Preparation of Premixes:
Premix 1:—76 g of demineralized water,
   76 g of cellulase enzyme solution (pH 6) containing
      2% by weight of cellulase (same as example 1),
      35% by weight of 1,2,3-trihydroxypropane (glycerol),
      20 mMol sodium citrate and
      0.15% by weight Proxel® GXL and
   9.72 g of 25% aq. sodium hydroxide solution
Premix 2: A premix of 186.92 g of isoparaffin (Isopar® G) oil and 21.64 g of amphiphilic polymer solution S1 was prepared.
Premix 3: A premix of 3.08 g of terephthaloyl chloride (TPC) and 27.64 g dibutyl adipate was prepared.

Synthesis was run according to the procedure described in Example 1.

Capsules size: d50=3.1 μm

### Example 3

Preparation of Premixes:
Premix 1:—76 g of demineralized water,
   76 g of cellulase enzyme solution (pH 6) containing
      2% by weight of cellulase (same as example 1),
      35% by weight of 1,2,3-trihydroxypropane (glycerol),
      20 mMol sodium citrate and
      0.15% by weight Proxel® GXL and
   9.72 g of 25% aq. sodium hydroxide solution
Premix 2: A premix of 186.92 g of isoparaffin (Isopar® G) oil and 21.64 g of amphiphilic polymer solution S1 was prepared.
Premix 3: A premix of 3.08 g of terephthaloyl chloride (TPC), 3.08 g of adipoyl chloride (ADC) and 55.28 g of dibutyl adipate was prepared.

Synthesis was run according to the procedure described in Example 1.

Capsules size: d50=2.8 μm

### Example 4

Preparation of Premixes:
Premix 1:—32 g of demineralized water,
   120 g of cellulase enzyme solution (pH 6) containing
      2% by weight of cellulase (same as example 1),
      35% by weight of 1,2,3-trihydroxypropane (glycerol),
      20 mMol sodium citrate and
      0.15% by weight Proxel® GXL and
   9.72 g of 25% aq. sodium hydroxide solution
Premix 2: A premix of 186.92 g of isoparaffin (Isopar® G) oil and 21.64 g of amphiphilic polymer solution S1 was prepared.
Premix 3: A premix of 6.16 g of terephthaloyl chloride (TPC) and 55.28 g dibutyl adipate was prepared.

Synthesis was run according to the procedure described in Example 1.

Capsules size: d50=3.4 μm

### Example 5

Preparation of Premixes:
Premix 1:—76 g of demineralized water,
   76 g of cellulase enzyme solution (pH 6) containing
      2% by weight of cellulase (same as example 1),
      35% by weight of 1,2,3-trihydroxypropane (glycerol),
      20 mMol sodium citrate and
      0.15% by weight Proxel® GXL and
   9.72 g of 25% aq. sodium hydroxide solution
Premix 2: A premix of 186.92 g of isoparaffin (Isopar® G) oil and 21.64 g of amphiphilic polymer solution S1 was prepared.
Premix 3: A premix of 6.16 g of adipoyl chloride (ADC) and 55.28 g dibutyl adipate was prepared.

Synthesis was run according to the procedure described in Example 1.

Capsules size: d50=4.0 μm

### Example 6

Preparation of Premixes:
Premix 1: 76 g of demineralized water,
   76 g of cellulase enzyme solution containing 2% by weight of cellulase (same as example 1), 20 mMol sodium citrate and 0.15% by weight Proxel® GXL
   2.8 g of 1,2,3-trihydroxypropane (glycerol) and
   9.72 g of 25% by weight aq. sodium hydroxide solution.
Premix 2: A premix of 186.92 g of isoparaffin (Isopar® G) oil and 21.64 g of amphiphilic polymer solution S1 was prepared.
Premix 3: A premix of 6.16 g terephthaloyl chloride (TPC) and 55.28 g dibutyl adipate was prepared.

Synthesis was run according to the procedure described in Example 1.

Capsules size: d50=2.9 μm

### Example 7

Preparation of Premixes:
Premix 1:—76 g of demineralized water
   76 g of cellulase enzyme solution containing 2% by weight of cellulase (same as example 1), 20 mMol sodium citrate and 0.15% by weight Proxel® GXL,
   6.97 g of 1,2,3-trihydroxypropane (glycerol) and
   9.72 g of 25% by weight aq. sodium hydroxide solution was prepared.
Premix 2: A premix of 186.92 g of isoparaffin (Isopar® G) oil and 21.64 g of amphiphilic polymer solution S1 was prepared.
Premix 3: A premix of 12.28 g of terephthaloyl chloride (TPC) and 55.28 g dibutyl adipate was prepared.

Synthesis was run according to the procedure described in Example 1.

Capsules size: d50=4.5 μm

### Example 8

Preparation of Premixes:
Premix 1:—76 g of demineralized water,
   76 g of cellulase enzyme solution containing 2% by weight of cellulase (same as example 1), 20 mMol sodium citrate and 0.15% by weight Proxel® GXL
   5.22 g of 1,2,3-trihydroxypropane (glycerol) and
   9.72 g of 25% aq. sodium hydroxide solution
Premix 2: A premix of 186.92 g of isoparaffin (Isopar® G) oil and 21.64 g of amphiphilic polymer solution S1 was prepared.
Premix 3: A premix of 9.21 g of terephthaloyl chloride (TPC) and 55.28 g dibutyl adipate was prepared.

Synthesis
Synthesis was run according to the procedure described in Example 1.

Capsules size: d50=3.9 μm

### Example 9

Preparation of Premixes:
Premix 1-76 g of demineralized water,
   76 g of cellulase enzyme solution containing 2% by weight of cellulase (same as example 1), 20 mMol sodium citrate and 0.15% by weight Proxel® GXL
   1.04 g of 1,2,3-trihydroxypropane (glycerol) and
   9.72 g of 25% by weight aq. sodium hydroxide solution
Premix 2 A premix of 186.92 g of isoparaffin (Isopar® G) oil and 21.64 g of amphiphilic polymer solution S1 was prepared.
Premix 3 A premix of 1.84 g of terephthaloyl chloride (TPC) and 55.28 g dibutyl adipate was prepared.

Synthesis

Synthesis was run according to the procedure described in Example 1.

Capsules size: d50=4.1 μm

### Example 10

Like Example 6, but 2,2-Bis(hydroxymethyl)1,3-propanediol was used instead of 1,2,3-trihydro-xypropane

Capsules size: d50=3.0 μm

### Example 11

Like Example 6, but polyvinyl alcohol having a degree of hydrolysis of ˜88% (Mowiol® 18-88) was used instead of 1,2,3-trihydro-xypropane.

Capsules size: d50=4.6 μm

### Example 12

Like Example 6, but adipic acid butyl ester was used instead of isoparaffin (Isopar® G) oil.

Capsules size: d50=7.2 μm

### Example 13

Like Example 6, but propylene glycol dicaprylate/dicaprate (Myritol® PC) was used instead of isoparaffin (Isopar® G) oil.

Capsules size: d50=0.9 μm

Application Tests

In the application tests the depolymerization of an aqueous solution of guar gum was studied by monitoring the viscosity of the aqueous solutions.

Rheology studies were performed using guar solutions and various microencapsulated enzymes in form of the microcapsule dispersion obtained by the examples 1, 2, 3 and 5. A control assay was performed with guar solution without any enzyme addition and a further control assay was performed with guar solution and enzyme which was not encapsulated. The tests were carried out on a Grace M5600 HPHT rheometer with temperature setting at 95° C. and pressure setting at 3.45*10⁶ Pa.

Guar gum was first hydrated in water from an oil slurry (POLYfrac® Plus M-4.5 from PfP Technology containing 540 kg/m³ of slurry in mineral oil) to a final guar concentration of 0.3% by wt. for 45 min by vigorous mixing (at 1000 rpm using a mixer or disperser). A surfactant solution (SHALE SURF® 1000 from Frac Tech Services International, containing a blend of ethoxylated alcohol, 2-butoxyethanol, 2-propanol, cyclohexene and methanol) and a clay stabilizer solution (KCLS-4 from Frac Tech Services International) were then added to the guar solution to final concentrations of 0.1% and 0.05%, respectively, with additional mixing for 1 minute. The pH of the guar solution was adjusted to 10.5 with a pH adjuster solution (B-10 from Frac Tech Services International, containing potassium carbonate and potassium hydroxide).

A borate-based cross-linker with delayed release was then added to the guar solution to a concentration of 0.1% (BXL-3 from Frac Tech Services International, containing borate salt, crystalline silica, quartz, potassium formate, hydrated aluminum-magnesium silicate, sodium sulfate, sodium chloride and water). The solution was mixed for 1 minutes.

Fifty milliliter of the guar solution was immediately transferred upon preparation to the sample cup of the rheometer at ambient temperature and atmospheric pressure. The enzyme was added to the solution in the cup at desired concentration. The sample cup was then sealed to start the test with onset of high pressure at 3.45*10⁶ Pa and high temperature at 95° C., and solution viscosity was continuously measured.

The guar samples were treated at following conditions:

a) (Control) no enzyme addition.
b) Unencapsulated enzyme solution: 65 μl of enzyme solution containing 0.6% cellulase for high-temperature applications (Seq. ID No. 2 of WO 2013/148163 A1) was added to the 50 ml guar solution.
c) Different microencapsulated enzymes: 120-125 μl of microencapsulated enzyme containing cellulase (from examples 1, 2, 3 and 5) was added to the 50 ml guar solution.

Tests in b) and c) contained same amount of active enzyme, at 0.0008% by. wt., in the guar solution.

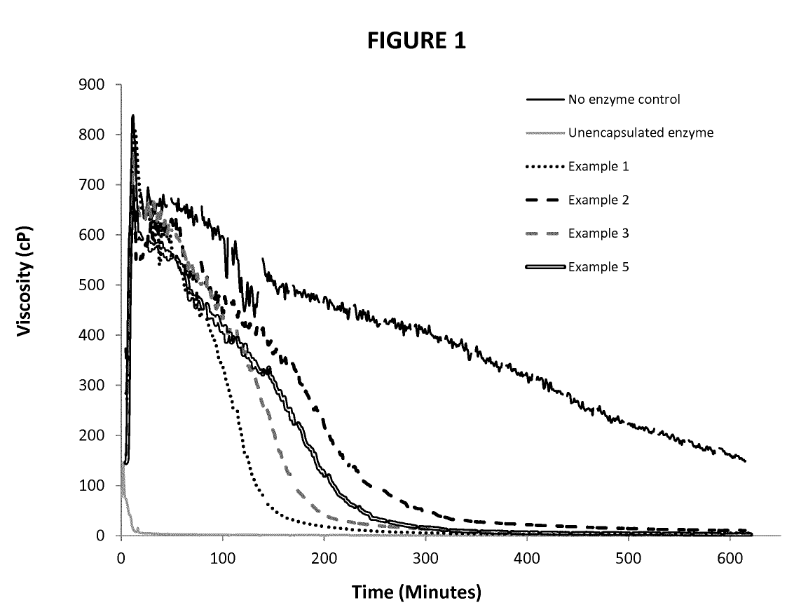

The results of these studies are shown in FIG. 1.

In the test without enzyme addition (a), the fluid viscosity showed slow and gradual destabilization over time, but maintained above 200 cP during the 10 hour test period. In the test with unencapsulated enzyme added (b), the fluid viscosity exhibited rapid reduction without any delay; actually, the viscosity did not even achieved the peak level as the other tests, and dropped below 10 cP within 15 minutes. The ending pH of this guar solution remained above pH 10.0.

In the tests with microencapsulated enzymes added (c), delay in viscosity reduction was clearly displayed with different delay times among different examples, and the ending pH of the solution also varied. In example 1, the viscosity decreased to baseline level after about 3 hours with an ending solution pH of 8.8; in example 2, the viscosity decreased to baseline level after about 6 hours with an ending solution pH of 8.4; in example 3, the viscosity decreased to baseline level after about 4 hours with an ending solution pH of 8.9; in example 5, the viscosity decreased to baseline level after about 5 hours with an ending solution pH of 9.2.

FIG. 1: Microencapsulated enzymes showed delayed profile in decreasing guar gum viscosity.

1.-21. (canceled)

22. Microcapsules with a shell and a core with an average particle size of the microcapsules in the range from 0.5 to 20 μm, wherein the shell is a polyester and wherein the core material comprises an enzyme and water.

23. The microcapsules according to claim 22, wherein the polyester is built by polycondensation of least one alcohol selected from the group consisting of diols and polyols and least one acid-component selected from the group consisting of divalent carboxylic acids, multivalent carboxylic acids, acid halides of a divalent carboxylic acid and acid halides of multivalent carboxylic acid.

24. The microcapsules according to claim 22, wherein the di- or polyol has 2 to 20 carbon atoms and at least two hydroxyl groups

25. The microcapsules according to claim 22, wherein the polyol is a polymeric polyol with a degree of polymerization (DP) from 10 to 6000.

26. The microcapsules according to claim 22, wherein the enzyme is a cellulase.

27. The microcapsules according to claim 22, which are obtainable by a process comprising the steps:
a) preparing an emulsion with an aqueous disperse phase, a hydrophobic continuous phase and a protective colloid,
wherein the aqueous disperse phase comprises the core material and at least one alcohol selected from the group consisting of diols and polyols, and
b) subsequently adding one or more acid halide of a di- or multivalent carboxylic acid
c) and polycondensation of the diol and/or polyol with the acid halide of a di- or multivalent carboxylic acid to build the microcapsule shell.

28. The microcapsules according to claim 27, wherein the protective colloid is an amphiphilic polymer.

29. A process for producing a dispersion of the microcapsules according to claim 22, comprising the process steps:
a) preparing an emulsion with aqueous disperse phase, an hydrophobic continuous phase and a protective colloid,
wherein the aqueous disperse phase comprises the core material and at least one alcohol selected from the group consisting of diols and polyols, and
b) subsequently adding one or more acid halide of a di- or multivalent carboxylic acid
c) and polycondensation of the diol and/or polyol with the acid halide of a di- or multivalent carboxylic acid to build the microcapsule shell.

30. A dispersion comprising 5 to 50% by weight, based on the total weight of the dispersion, of the microcapsules according to claim 22.

31. A process for reducing the viscosity of subterranean treatment fluids which comprises utilizing the microcapsules according to claim 22.

32. An aqueous fracturing fluid, wherein the aqueous fracturing fluid comprises
A) an aqueous base fluid,
B) a proppant,
C) a viscosifier, and
D) the microcapsules according to claim 22.

33. The aqueous fracturing fluid according to claim 32, wherein the viscosifier C) is a polymeric viscosifier, which comprises at least one polysaccharide and/or polysaccharide derivative, and wherein the microcapsules D) comprise at least a cellulase.

34. The aqueous fracturing fluid according to claim 32, wherein the viscosifier C) is a polymeric viscosifier, which comprises at least one guar gum and/or a guar gum derivative and a crosslinker, the microcapsules D) comprise at least a cellulase.

35. The aqueous fracturing fluid according to claim 32, wherein the viscosifier C) is a polymeric viscosifier, which comprises at least one guar gum and/or a guar gum derivative and a boron-containing crosslinker, the microcapsules D) comprise at least a cellulase, and wherein the pH value of the aqueous fracturing fluid is from 9.5 to 12.0.

36. A method of treating a subterranean formation, comprising contacting a subterranean formation with an aqueous treatment fluid, wherein the treatment fluid comprises the microcapsules according to claim 22.

37. A method of fracturing a subterranean formation, which at least comprises the steps of
(1) formulating an aqueous fracturing fluid,
(2) pumping the fracturing fluid down the wellbore at a rate and pressure sufficient to flow into the formation and to initiate or extend fractures in the formation,
(3) reducing the applied pressure thereby allowing at least a portion of the injected fracturing fluid to flow back from the formation into the wellbore, and
(4) removing such flowed back fracturing fluid from the wellbore,
wherein the aqueous fracturing fluid comprises at least
A) an aqueous base fluid,
B) a proppant,
C) a viscosifier, and
D) the microcapsules according to claim 22.

38. The method according to claim 37, wherein the viscosifier C) is a polymeric viscosifier, which comprises at least one polysaccharide and/or polysaccharide derivative, and wherein the microcapsules D) comprise at least a cellulase.

39. The method according to claim 37, wherein the viscosifier C) is a polymeric viscosifier, which comprises at least one guar gum and/or a guar gum derivative and a crosslinker, the microcapsules D) comprise at least a cellulase.

40. The method according to claim 37, wherein the viscosifier C) is a polymeric viscosifier, which comprises at least one guar gum and/or a guar gum derivative and a boron-containing crosslinker, the microcapsules D) comprise at least a cellulase, and wherein the pH value of the aqueous fracturing fluid is from 9.5 to 12.0.

41. The method according to claim 37, wherein the formation temperature is from 60° C. to 130° C.
